(19) United States
(12) Patent Application Publication
(10) Pub. No.: US 2023/0279393 A1
(43) Pub. Date: Sep. 7, 2023

(54) Treatment of Airway Conditions by Modulation of MiR200 Family MicroRNAs

(71) Applicant: The Regents of the University of California, Oakland, CA (US)

(72) Inventors: PRESCOTT WOODRUFF, San Francisco, CA (US); KRISTINA JOHANSSON, San Francisco, CA (US); SANA SIDDIQUI, San Francisco, CA (US)

(73) Assignee: The Regents of the University of California, Oakland, CA (US)

(21) Appl. No.: 17/769,322

(22) PCT Filed: Oct. 14, 2020
(86) PCT No.: PCT/US2020/055608

§ 371 Date: Apr. 14, 2022

Related U.S. Application Data
(60) Provisional application No. 62/915,098, filed on Oct. 15, 2019.

(51) Int. Cl. C12N 15/113; A61P 11/00

(52) U.S. Cl. CPC C12N 15/113; A61P 11/00; C12N 2310/113; C12N 2320/30

(57) ABSTRACT

The inventions provide the art with novel treatments of various airway conditions such as asthma wherein pathological production of mucus is implicated. Disclosed are novel inhibitors of miR-200 micro-RNAs, including inhibitors of miR-141. These miRNAs promote pathological mucus production and other airway dysfunction. They may be targeted by antagomirs which disrupt their activity. Additionally, they may be targeted by compositions with disrupt the gene expression of the targeted miR.

CROSS-REFERENCE TO RELATED APPLICATIONS: This application is a §371 national stage filing of PCT Application Number PCT/US2020/055608, entitled “Treatment of Airway Conditions by Modulation of MiR200 Family MicroRNAs,” filed Oct. 14, 2020, which application claims the benefit of priority to U.S. Provisional Pat. Application Serial No. 62/915,098 entitled “Treatment of Airway Conditions by Modulation of MiR200 Family MicroRNAs,” filed Oct. 15, 2019, the contents of which applications are hereby incorporated by reference.

STATEMENT REGARDING FEDERALLY SPONSORED RESEARCH OR DEVELOPMENT: This invention was made with government support under grant number U19 AI077439 awarded by the National Institutes of Health. The government has certain rights in the invention.

The instant application contains a Sequence Listing which has been submitted electronically in ASCII format and is hereby incorporated by reference in its entirety. Said ASCII copy, created on Oct. 14, 2020, is named UCSF083PCT_SL.txt and is 5,364 bytes in size.

### BACKGROUND OF THE INVENTION

Airway conditions such as T2-high asthma are defined by chronic type 2 inflammation in the airway which causes bronchial hyper-reactivity and airflow obstruction. Epithelial cells form a barrier to the external environment and secrete mucus that traps inhaled particles and pathogens. Defective epithelial function is a defining feature of asthma and increased production of pathological mucus by airway epithelial cells can lead to mucus plugs that limit airflow and accumulate in asthma exacerbations.

Airway goblet cells develop from basal cells and are specialized to produce, store and release mucins, and thereby play a major role in airway plugging. Despite the importance of mucus production in the pathophysiology of asthma and other respiratory diseases, there are currently no effective therapies that specifically target mucus overproduction in the airway.

Many people with asthma display evidence of a T2-high phenotype with atopy and ongoing type 2 airway inflammation mediated by the cytokines interleukin (IL)-4, IL-5 and IL-13. While IL-4 and IL-5 drive immunoglobulin (Ig) E production and eosinophilia, respectively, IL-13 has important effects on structural cells including airway epithelial cells. IL-13 signaling through STAT6, subsequent engagement of the transcription factor SPDEF (SAM pointed domain-containing Ets transcription factor), and alterations in the balance of FOXA2/FOXA3 (forkhead box A2/A3) are critical steps in a major pathway for airway epithelial goblet cell metaplasia. This pathway preferentially induces the mucin glycoprotein MUC5AC (as compared to MUC5B) in vitro, recapitulating its preferential induction of MUC5AC in airway epithelial brushings from humans with T2-high asthma. MUC5AC may be particularly pathological as it is poorly transported by the mucociliary apparatus and is the predominant mucin glycoprotein in fatal asthmatic airway plugs.

Micro RNAs (miRNAs) represent a distinct class of noncoding RNAs, about ~20-22 nucleotides long, that mediate sequence-specific repression of target mRNAs, inhibiting gene expression at the post-transcriptional level. The seed sequence, situated at positions 2-7 from the miRNA 5′-end, mediates target recognition at sites typically within the 3′UTR of messenger RNAs (mRNAs). The most powerful and defining feature of miRNAs is their ability to regulate multiple target genes with related cellular functions. Thus, a single miRNA can have a major biological impact by acting as a master regulator of several genes in an inflammatory pathway. It has previously been reported that airway miRNA expression, including miR-141 expression, may differ in asthmatics compared to healthy controls, for example, as described in Solberg et al., 2012. Airway Epithelial miRNA Expression Is Altered in Asthma. Am JRespir Crit Care Med. 186:965-74. Some miRNAs have been studied in relation to differentiation of ciliated cells, but published studies focusing on miRNAs related to mucus production are limited. miR-141 belongs to the miR-141/200 family. miR-141 has not been reported to have a direct role in mucus production in asthma, however it has a number of predicted mucus- related targets including FOXA2, for example, as described in Li et al., 2018. microRNA-141-3p fosters the growth, invasion, and tumorigenesis of cervical cancer cells by targeting FOXA2. Arch Biochem Biophys. 657:23-30. LacZ reporter expression for murine miR-141 revealed a remarkable expression pattern that is observed almost exclusively in the adult murine airway, including the nasal cavity, trachea, bronchi, and bronchioles, although it is also present in the olfactory bulbs of the brain, for example, as described in Park CY, et al., 2012. A Resource for the Conditional Ablation of microRNAs in the Mouse. Cell Rep. 1:385-91. This observation highlights that miR-141 is abundantly, and specifically, expressed in the branching airway, however, the role of this micro RNA in airway health is unknown.

Accordingly, there remains a need in the art for an improved understanding of the regulation of mucus production in airway conditions such as asthma. There remains a need in the art for novel treatments of asthma and other airway conditions wherein mucus production is implicated. These needs and others are addressed by the disclosures of the present invention.

### SUMMARY OF THE INVENTION

The scope of the invention encompasses various methods for the treatment of asthma, pathological mucus production, and other airway conditions by the modulation of miR-200 family miRNAs. As disclosed herein, miR-200 miRNAs are implicated in numerous pathological airway processes. As demonstrated herein, modulation of miR-200 family micro-RNAs inhibits pathological processes and provides a means of prevention and treatment for many airway conditions.

In a first aspect, the scope of the invention encompasses a method of treating an airway condition associated with impaired airway function, including pathological mucus production by airway cells. In a primary implementation, the airway condition is asthma, for example, high Th2 asthma.

The various methods of the invention encompass the administration of an agent which modulates the activity one or more miR-200 family members in airway cells. In one embodiment, the miR-200 family member is miR-141. In one aspect, the modulation of the miR-200 family member encompasses inhibition, for example, inhibition of miR-141.

In a primary implementation, the scope of the invention encompasses an inhibitor of one or more miR-200 miRNAs. In one embodiment, the inhibitor is an inhibitor of miR-141. The inhibition of miR-200 miRNAs may be achieved by any number of agents. In a first implementation, the miR-200 miRNA inhibitor is an antagomir or like construct which hybridizes to RISC-associated target miRNAs, blocking their repressive activity. In other implementations, the inhibitor may comprise an agent which reduces the abundance of the targeted miR-200 miRNA by inhibiting the expression of genes thereof, or by disrupting the post-translational processing of the targeted miRNA.

The novel agents and treatments of the invention may advantageously be administered by an inhalation, intratracheal, or intranasal route, providing a direct and selective treatment of airway cells that avoids off-target effects.

The methods and associated agents of the invention provide the art with novel means of treating various pathological processes in airway cells and treating airway conditions such as asthma. These and other benefits are disclosed in the detailed description of the invention which follows.

### BRIEF DESCRIPTION OF THE DRAWINGS

FIGS. 1A and 1B. miR-141 is abundantly expressed in the human airway epithelium. FIG. 1A depicts the 40 most highly expressed miRNAs in bronchial epithelial brushings. miR-141/200 family miRNAs (141/200a/200c/429) are highlighted by black bars. FIG. 1B: Microarray analysis of hsa-miR-141-3p in human epithelial brushings from mild asthmatics (not using inhaled corticosteroids) and moderate asthmatics (using inhaled corticosteroids) compared to healthy controls (n=12-16/group, One-way ANOVA with Dunnett’s multiple comparison test, ****P<0.0001)..

FIGS. 2A, 2B, 2C, and 2D. CRISPR/Cas9-mediated knockdown of miR-141 in primary HBECs grown at air-liquid interface. FIG. 2A: Mature miRNA sequences and genomic location of the miR-141/200 family in humans and mice (SEQ ID NOS 1, 18, 3, 19, 7, 21, 5, 20, 9 and 22, respectively, in order of appearance). FIG. 2B: Electroporation (EP)-based CRISPR/Cas9-protocol established in an in vitro ALI system (day 0-28, +/- IL-13 day 21-28) using HBECs. FIG. 2C: Expression level of hsa-miR-141-3p by TaqMan qPCR following administration of MIR141-targeting versus non-targeting (NT) gRNAs normalized to reference (ref) miRNAs hsa-miR-103a-3p and hsa-miR-191-5p (n=8, two-tailed t-test, ***P<0.001). FIG. 2D: Correlation of MIR141-targeting efficiency score assessed by Sanger DNA sequencing and ICE Synthego analysis and hsa-miR-141-3p expression levels by qPCR. rP, Pearson correlation coefficient.

FIGS. 3A, 3B, 3C and 3D. CRISPR/Cas9-targeting of miR-141 reduces IL-13-induced mucus. FIGS. 3A and 3B: MUC5AC mean fluorescent intensity (MFI) (n=9, two-tailed paired t-test, *P<0.05, **P<0.01). FIG. 3C: Quantification of mucus-producing cells in secreted MUC5AC assessed by dot blot analysis of apical wash FIG. 3D: Quantification of mucus-producing cells in ALI-cultured HBECs following NT or MIR141 gRNA delivery (n=3- 7/group, One-way ANOVA followed by the Holm-Sidak test, *P<0.05, ***P<0.001).

FIGS. 4A and 4B. miR-141 repression in gene-edited airway epithelial cells is associated with a reduction in mucus-producing goblet cell numbers. FIG. 4A: Frequency of ciliated cells, basal cells, TSPAN8⁻ secretory cells and TSPAN8⁺ secretory cells (green) (% of all cells, n=4/group) in ALI-cultured NT or MIR141-targeted HBECs with (IL-13) or without (UT) IL-13 (two-tailed paired t-test). FIG. 4B: Hsa-miR-141-3p expression fold difference assessed by TaqMan qPCR in FACS sorted ciliated cells (SiR-tubulin⁺NGFR⁻) basal cells (SiR-tubulin⁻NGFR⁺) and TSPAN8⁻ secretory cells compared to IL-13-inducible TSPAN8⁺ secretory cells.

FIGS. 5A, 5B, 5C, 5D, 5E, 5F, 5G, 5H, and 5I. Blockade of mmu-miR-141-3p improves airway hyper-responsiveness and decreases secreted mucus in an experimental mouse model of asthma. FIG. 5A: Timeline of allergen-induced model of asthma induced by intranasal (i.n.) exposure to fungal allergen Aspergillus fumigatus (Aps). FIG. 5B: Total cells. FIG. 5C: Cellular distribution (Eos, eosinophils; Neu, neutrophils; Lym, lymphocytes; Mac, macrophages) in bronchoalveolar lavage (BAL) obtained from mice exposed to Asp in combination with mmu-miR-141-3p antagomir (Asp/miR-141 inhib), Asp in combination with scrambled antagomir (Asp/Scr) and sterile saline in combination with Scr antagomir (Sal/Scr) (n=7-8/group, two-way ANOVA followed by Dunnett’s test, *P<0.05, **P<0.01, ****P<0.0001). FIG. 5D: Total respiratory system resistance and elastance measured in mice exposed to Asp/miR-141 inhib, Asp/Scr and Sal/Scr (n=7-8/group, repeated measures ANOVA followed by Bonferroni correction, **P<0.01, ***P<0.001, ****P<0.0001). FIG. 5E: Gene expression of Muc5ac assessed by qPCR analysis of lung tissue homogenate 72 h after the final allergen challenge (n=4/group, one-way ANOVA followed by the Tukey test, *P<0.05). FIG. 5F: Gene expression of Clca1 assessed by qPCR analysis of lung tissue homogenate 72 h after the final allergen challenge (n=4/group, one-way ANOVA followed by the Tukey test, *P<0.05). FIG. 5G, Representative Alcian Blue-Periodic Acid Schiff (AB-PAS)- stained lung sections from Asp/miR-141 inhib, Asp/Scr and Sal/Scr mice. Quantification of PAS⁺ cells per perimeter of basal membrane in large (>0.80 mm) and small (<0.80 mm) airways (n=7-8/group, one-way ANOVA). FIG. 5H: Representative intraluminal mucus per basal membrane from Asp/miR-141 inhib, Asp/Scr and Sal/Scr mice in large (>0.80 mm) and small (<0.80 mm) airways (n=7-8/group, one-way ANOVA).

FIG. 6. CRISPR gRNA design for targeted knockdown of MIR141 gene. FIG. 6 depicts the human MIR141gene (SEQ ID NO: 23). The map denotes the location of the stem loop, mature hsa-miR-141-5p and hsa-miR-141- 3p regions within the stem loop and three CRISPR guide RNAs: location of RNA guide sequences gRNA-1 (SEQ ID NO: 15), gRNA-2 (SEQ ID NO: 16), and gRNA-3 (SEQ ID NO: 17).

### DETAILED DESCRIPTION OF THE INVENTION

The scope of the invention encompasses various methods of improving airway function and treating airway conditions by modulation of one or more miRNAs of the MiR-200 family. In a primary embodiment, the scope of the invention encompasses various methods of improving airway function and treating airway conditions by the inhibition of miR-141. In a related aspect, the scope of the invention encompasses novel modulators of one or more other miRNAs of the MiR-200 family, for use in methods of improving airway function or treating an airway condition.

MiR-200 miRNAs. As used herein, a “miR-200 miRNA” or “member of the miR-200 family” is a micro RNA of the miR-200 family. The miR-200 family encompasses five members, as follows.

miR-141. “miR-141,” as used herein, may refer to any form of miR-141. In a primary embodiment, miR-141 refers to miR-141-3p. In another embodiment, miR-141 refers to miR-141-5p. In a primary embodiment, miR-141 refers to human sequences of miR-141, including hsa-miR-141-3p (uaacacugucugguaaagaugg; SEQ ID NO: 1) and hsa-miR-141-5p (caucuuccaguacaguguugga; SEQ ID NO: 2). In alternative embodiments, miR-141-3p and miR-141-5p sequences from other species are addressed, for example, sequences from mice, rats, canines, felines, horses, pigs, cows, non-human primates, and other animal species.

miR-200a. miR-200a, as used herein, may refer to any form of miR-200a. In one embodiment, miR-200a refers to miR-200a-3p. In another embodiment, miR-200a refers to miR-200a-5p. In a primary embodiment, miR-200a refers to human sequences of miR-200a, including hsa-miR-200a-3p (uaacacugucugguaacgaugu; SEQ ID NO: 3) and hsa-miR-200a-5p (caucuuaccggacagugcugga; SEQ ID NO: 4). In alternative embodiments, miR-200a-3p and miR-200a-5p sequences from other species are utilized.

miR-200b. miR-200b, as used herein, may refer to any form of miR-200b. In one embodiment, miR-200b refers to miR-200b-3p. In another embodiment, miR-200b refers to miR-200b-5p. In a primary embodiment, miR-200b refers to human sequences of miR-200b, including hsa-miR-200b-3p (uaauacugccugguaaugauga; SEQ ID NO: 5) and hsa-miR-200b-5p (caucuuaccggacagugcugga; SEQ ID NO: 6). In alternative embodiments, miR-200b-3p and miR-200b-5p sequences from other species are utilized.

miR-200c. miR-200c, as used herein, may refer to any form of miR-200c. In one embodiment, miR-200c refers to miR-200c-3p. In another embodiment, miR-200c refers to miR-200c-5p. In a primary embodiment, miR-200c refers to human sequences of miR-200c, including hsa-miR-200c-3p (uaauacugccggguaaugaugga; SEQ ID NO: 7) and hsa-miR-200c-5p (cgucuuacccagcaguguuugg; SEQ ID NO: 8). In alternative embodiments, miR-200c-3p and miR-200a-5c sequences from other species are utilized.

miR-429. miR-429, as used herein, may refer to any form of miR-429. In one embodiment, miR-429 refers to human sequences of miR-429 (uaauacugucugguaaaaccgu; SEQ ID NO: 9).

The methods of the invention encompass the modulation of one or more miRNAs of the miR-200 family. “Modulation,” as used herein encompasses any change, e.g. increase or decrease, in the expression, biological activity, or abundance of the selected miR-200 miRNA, including changes in the temporal or spatial patterns of expression. In a primary embodiment, modulation encompasses inhibition. As used herein, “inhibition” may encompass any reduction in the expression, processing to maturity, abundance, biological activity, or lifespan of a selected miRNA. In various embodiments, inhibition may encompass, for example, at least 10%, at least 20%, at least 30%, at least 40%, at least 50% at least 60%, at least 70%, at least 80%, or at least 90% reduction in the selected measure, for example, measured relative to like untreated cells or organisms.

Various implementations of the invention encompass the treatment of an airway condition. As used herein, “treatment” may encompass any therapeutic effect with respect to a selected pathological process or condition. Treatment may encompass reducing the severity, ameliorating the symptoms, inhibiting the underlying cause of, slowing or halting the progression of, preventing, curing, reducing the morbidity of, reducing the probability of mortality from, or otherwise therapeutically affecting a selected condition or pathological process. In various embodiments, of the invention, treatment may encompass reducing or preventing mucus overproduction in airway cells; inhibiting the formation of mucus plugs; improving defective airway epithelial function; improving airway function, including function as assessed FeNO, spirometry, and peak flow testing; reducing the symptoms of asthma, including Th2 high asthma; reducing the effects of allergies; reducing airway inflammation; inhibiting airway hyperresponsiveness; reducing IL-13 mediated processes; reducing epithelial goblet cell metaplasia; and/or reducing mucin glycoprotein MUC5AC production.

Various implementations of the inventions disclosed herein encompass the treatment of a selected airway condition. As used herein, an “airway condition” encompasses any pathological, dysregulated, aberrant, or impaired airway function. Exemplary airway conditions include, for example, impaired airway function, for example, as assessed by spirometry, peak flow testing, or FeNO testing; mucus overproduction in airway cells; formation of mucus plugs; defective airway epithelial function; asthma; allergies; airway inflammation; airway hyperresponsiveness; a lung disease; chronic obstructive pulmonary disease; cystic fibrosis; pathological IL-13 mediated processes; epithelial goblet cell metaplasia; and mucin glycoprotein MUC5AC overproduction. In a primary embodiment, the airway condition is asthma, including asthma associated with high Th-2 responses, for example, asthma associated with elevated Th-2 helper cell derived cytokines including IL-4, IL-5, IL-9, and IL-13. In some aspects, the airway condition is asthma associated with airway function impairment and obstruction by mucus overproduction. In some aspects, the asthma is asthma associated with exposure to allergens. In some aspects, the airway condition is chronic cough. In some aspects, the airway condition is cough associated with infection. In some aspects the airway condition is chronic sinus disease, for example, chronic rhinosinusitis or chronic rhinosinusitis with nasal polyps.

Various methods of the invention encompass the delivery of therapeutic compositions to airway cells. As used herein, “airway cells” encompass any cells of the airway, including cells extending from the nasal passages to the trachea to the lungs. Airway cells may include airway epithelial cells, smooth muscles, fibroblasts, and endothelial cells. A primary target comprises the epithelial cells which line the, including in the airway nasal cavity, trachea, bronchi, and bronchioles.

In various implementations, the methods disclosed herein may encompass the administration of a therapeutically effective amount of a selected agent. As used herein, “a therapeutically effective amount” is an amount sufficient to promote a measurable biological effect, including measurable therapeutic effects.

The various implementations of the inventions disclosed herein may encompass the administration of agents to a subject. As used herein, a “subject” may encompass any animal, for example, a human, such as a human patient. Subjects may further include non-human animals such as test animals, livestock, pets, and veterinary subjects, such as mice, rats, felines, canines, non-human primates, and other species.

Various implementations of the invention encompass nucleic acid sequences. In some embodiments, the invention encompasses subsequences and variants of an enumerated sequence. As used herein, a “subsequence” comprises a subset of the enumerated sequence. In a primary embodiment, the subsequence comprises a continuous subsequence of the enumerated sequence. In other embodiments, the subsequence comprises a discontinuous subset of the enumerated sequence, for example, a subsequence comprising one or more interruptions. In various embodiments, the subsequence may comprises at least five, six, seven, eight, nine, ten, eleven, twelve, thirteen, fourteen, fifteen, sixteen, seventeen, eighteen, nineteen, twenty, or 21 nucleotides of the enumerated sequence. In one embodiment, the subsequence comprises 16-21 nucleotides of the enumerated sequence, in one embodiment the subsequence being a continuous subsequence. In one embodiment, the subsequence comprises 16-20 nucleotides of the enumerated sequence, of the enumerated sequence, in one embodiment the subsequence being a continuous subsequence. In one embodiment, the subsequence comprises 16-18 nucleotides of the enumerated sequence, of the enumerated subsequence, in one embodiment the subsequence being a continuous subsequence.

In some implementations encompassing the use of an enumerated nucleic acid sequence, the scope of the invention encompasses variants of the enumerated sequence. In some embodiments, variant of the enumerated sequence comprises a sequence with one, two, three, or more nucleotide substitutions, mismatched nucleotides, deleted nucleotides, or added nucleotides, relative to the enumerated sequence. In one embodiment, the variant comprises one such mismatched nucleotides, deleted nucleotides, or added nucleotides, relative to the enumerated sequence. In one embodiment, the variant comprises two such mismatched nucleotides, deleted nucleotides, or added nucleotides, relative to the enumerated sequence. In other implementations, the variant comprises a sequence of at least 70%, at least 75%, at least 80%, at least 90%, at least 95%, or at least 99% sequence identity to the enumerated sequence.

miR-200 miRNA Inhibitors. In a first aspect, the scope of the invention encompasses compositions of matter which act as a modulator of one or more miR-200 miRNAs. In a primary aspect, the modulator of one or more miR-200 family miRNAs is an inhibitor of the one or more miR-200 miRNAs. In one embodiment, the inhibitor is an inhibitor of miR-141. The inhibitors of the invention may comprise any composition of matter which inhibits its miRNA target, by any mechanism. In a primary implementation, the inhibitor acts by binding to the target miRNA and reducing its activity. In other implementations, the inhibitor reduces the abundance of the target miRNA by reducing its expression or disrupting its post-transcriptional processing.

Antagomirs. In one implementation, the miR-200 miRNA inhibitor is an antagomir. An antagomir, as used herein, means a composition of matter which binds one or more target miR-200 miRNAs and inhibits its biological activity, i.e., repression of target genes regulated by the one miRNA. Also known as and antimir or blockmir, antagomirs are nucleic acid compositions comprising a sequence complementary to target miRNA. miRNAs achieve suppression of target genes by RNA interference. Mature micro-RNAs, typically of 20-22 nucleotides in length, are processed in the cytoplasm from pre-miRNAs exported from the nucleus, which in turn are produced from longer pri-miRNA transcripts. Mature, single-stranded micro-RNAs are loaded into and incorporated within the RNA-induced silencing complex, or(RISC), which is a ribonucleoprotein complex comprising the endonuclease Argonaute 2. The integrated miRNA binds with high selectivity and affinity to complementary sequences in the 3′ UTRs of target messenger RNA transcripts. This binding activates Argonaute, resulting in the cleavage and subsequent degradation of the targeted messenger RNA. Binding of antagomirs to the miRNA loaded in the RISC will competitively inhibit RISC binding to target mRNAs, essentially blocking access to target mRNAs.

The antagomirs of the invention may comprise nucleotides, modified nucleotides, nucleotide analogs, and mixtures of the foregoing. The compositions may be selected for improved binding affinity for target miRNA’s and/or increased resistance to nucleases and other degradation mechanisms.

In various embodiments, the micro-RNA binding compositions of the invention may comprise DNA, RNA, or nucleoside analogs and modified forms thereof. In some embodiments, the micro-RNA binding composition comprises a peptide nucleic acid, for example N-(2aminoethyl)-glycine.

In various embodiments, the antagomir will comprise nucleotides comprising modifications of the nucleotide sugar backbone, modifications of the nucleobase element of the nucleotide, or modifications of the linkage between nucleotides. In some implementations, the antagomir comprises one or more nucleotides having 2′-O-methyl (2′-O-Me), 2′-O-methoxyethyl (2′-MOE) and 2′-fluoro (2′-F) modified sugar moieties of the nucleotides, which confers increased nuclease resistance and may improve affinity for target micro-RNAs.

In some implementations, the antagomirs of the invention comprise one or more locked nucleic acids, wherein the ribose of RNA is modified with a methylene bridge between the 2′ oxygen and 4′ carbon.

In some embodiments, the antagomirs of the invention comprise morpholinos, comprising one or more six-membered morpholine ring.

In some embodiments, the antagomirs of the invention comprise one or more internucleotide linkages comprising a phosphorothioate (PS) linkage, wherein a sulfur replaces one of the oxygen atoms in the linking phosphate group. In various embodiments, the antagomir comprises one or more PS linkages selected from the group consisting of the linkage between the first and second nucleotide, the linkage between second the and third nucleotide, the linkage between the 18^{th} and 19^{th} nucleotide; the linkage between 19^{th} and 20^{th} nucleotide, the linkage between the 20^{th} and 21^{st} nucleotide, and the linkage between the 21^{st} and 22^{nd} nucleotide.

In one embodiment, antagomirs of the invention comprises one or more modifications to improve cellular uptake. For example, in one embodiment, the modification comprises a terminal cholesterol moiety, for example, a 3′-conjugated cholesterol molecule.

In a primary implementation, the antagomir comprises a sequence complementary to all of the nucleotides of the selected micro-RNA target. In other embodiments, the antagomir comprises a subsequence, being complementary to some of the target micro-RNA target, for example, wherein subsequence comprises a sequence complementary to at least five, six, seven, eight, nine, ten, eleven, twelve, thirteen, fourteen, fifteen, sixteen, seventeen, eighteen, nineteen, twenty, or 21 continuous or non-continuous nucleotides of the target miRNA. In some embodiments, the antagomir sequence comprises perfect complementarity to the targeted micro-RNA sequence. In some embodiments, the antagomir comprises one or more mismatches between its sequence and the complementary region of the targeted micro-RNA, for example, one two, three, or more mismatches.

In some embodiments, the antagomir comprises an polynucleotide or other miRNA binding composition, in some embodiments, comprising nucleic acids, modified nucleic acids, nucleotide analogs, or mixtures of the foregoing, sequence selected from SEQ ID NO: 10, SEQ ID NO: 11, SEQ ID NO: 12, SEQ ID NO: 13, or SEQ ID NO: 14, as described below. In some embodiments, the antagomir comprises a subsequence of the sequence selected from SEQ ID NO: 10-14. In some embodiments, the antagomir comprises a variant of a sequence selected from SEQ ID NO: 10-SEQ ID NO: 14.

In some embodiments, the antagomir comprises a sequence that will bind to the seed region of a targeted miR-200 family miRNA, or multiple miRNAs of the miR-200 family. In some embodiments, the micro-RNA binding composition comprises 5-8 nucleotides of the targeted seed region. In some embodiments, the micro-RNA binding composition of the invention comprises a tiny LNA, as known in the art, comprising a seed region-targeting sequence.

In a primary implementation, the antagomir is a monomer comprising a single sequence that hybridizes to the target miRNA. In alternative embodiments, the antagomir comprises two or more miRNA targeting sequences, for example a linear or circular molecule comprising multiple miRNA binding sites.

In a primary embodiment, the antagomir is an inhibitor of miR141. In one embodiment, the inhibitor of miR-141 is an inhibitor of miR-141-3p, for example, hsa-miR-141-3p. In one embodiment, the antagomir comprises SEQ ID NO: 10: ccaucuuuaccagacaguguua, or a subsequence or variant thereof In some embodiments, the antagomir comprises SEQ ID NO: 10 wherein any or all of: the linkage between the first and second nucleotide; the linkage between second the and third nucleotide; the linkage between the 18^{th} and 19^{th} nucleotide; the linkage between 19^{th} and 20^{th} nucleotide; the linkage between the 20^{th} and 21^{st} nucleotide; and the linkage between the 21^{st} and 22^{nd} nucleotide comprises a PS linkage. In one embodiment, the antagomir comprises SEQ ID NO: 10 or subsequence or variant thereof, wherein one or more, or all, nucleotides comprises 2-methoxy nucleotides. In one embodiment, the antagomir comprises SEQ ID NO: 10, or a subsequence of variant thereof, wherein the 3 ′, 5 ′ or both 3 ′ or 5 ′ ends are conjugated to a cholesterol moiety. In other embodiments, the antagomir is an antagomir of miR-141-5p, for example, an antagomir of hsa-miR-141-5p.

In some implementations, antagomir of the invention comprises an antagomir of miR-200a. In some embodiments, the antagomir as an inhibitor of miR-200a-3p. In some embodiments, the antagomir is an inhibitor of hsa-miR-200a-3p. In some embodiments the antagomir comprises SEQ ID NO: 11: acaucguuaccagacaguguua. In some embodiments, the antagomir as an inhibitor of miR-200a-5p. In some embodiments, the antagomir is an inhibitor of hsa-miR-200a-5p.

In some embodiments, the antagomir is an inhibitor of hsa-miR-200b. In some embodiments, the antagomir as an inhibitor of miR-200b-3p. In some embodiments, the antagomir is an inhibitor of hsa-miR-200b-3p. In some embodiments the antagomir comprises SEQ ID NO: 12: ucaucauuaccaggcaguauua. In some embodiments, the antagomir as an inhibitor of miR-200b-5p. In some embodiments, the antagomir is an inhibitor of hsa-miR-200b-5p.

In some embodiments, the antagomir is an inhibitor of miR-200c. In some embodiments, the antagomir as an inhibitor of miR-200c-3p. In some embodiments, the antagomir is an inhibitor of hsa-miR-200c-3p. In some embodiments the antagomir comprises SEQ ID NO: 13: uccaucacccggcaguauua. In some embodiments, the antagomir as an inhibitor of miR-200c-5p. In some embodiments, the antagomir is an inhibitor of hsa-miR-200c-5p.

In some embodiments, the antagomir is an inhibitor of miR-429. In some embodiments, the antagomir as an inhibitor of hsa-miR-429. In some embodiments the antagomir comprises SEQ ID NO: 14: acg guu cca gac agu auua.

Other Inhibitors. The scope of the invention further encompasses additional inhibitors of the activity miR-200 miRNAs. The inhibitors may comprise any composition that inactivates by binding, altering, or interferes with the activity of the targeted miRNA. Additional inhibitors may include antibodies, antigen-binding fragments thereof, aptamers, small molecules, and other compositions.

miR-200 Expression Inhibitors. In another aspect, the miR-200 modulators comprise compositions of matter that reduce the abundance of the targeted miRNA by disrupting the expression thereof, including by gene knockout, gene knockdown, RNA interference, or other mechanisms that disrupt the production of the targeted miRNA. In a primary implementation, the miR-200 Expression Inhibitor comprises a nucleic acid sequence which guides the selective mutation, deletion, or other inactivation of the target gene.

In a primary implementation, the miR-200 expression inhibitor is an element of a CRISPR-Cas9 or like system for the targeted knockdown of one or more genes of the miR-200 family. In one embodiment, the miR-200 gene inactivator comprises the 5 ′ targeting sequence of a CRISPR guide RNA, the targeting RNA comprising, for example, a 15-25 nucleotide subsequence of an miR-200 family member gene (either coding or non-coding strand), for example, a 17-20 nucleotide sequence, wherein the sequence is adjacent to a suitable protospacer adjacent motif (PAM site), for example, NGG, or CCN, wherein N is any nucleotide. In one embodiment, the guide sequence is present in an expression vector, such as a plasmid, which codes for the guide RNA sequence, and typically will be co-express an engineered Cas9 protein, for example, a Streptococcus pyognes Cas9 system (combined cRNA:tracrRNA, for example), for example, codon optimized for expression in the target organism, for example, optimized for expression in human cells. SpCas9 variants may also be used with altered PAM site specificities, for example, the D1135E, VRQ, EQR, VRER, xCas9, SpG and SpRY variants, as known in the art. The Cas9 and guide RNA sequences may be placed under the control of a suitable promoter. In one embodiment, the promoter is a promoter for selective or preferential expression in epithelial cells, airway cells, e.g. goblet cells, etc. When expressed in the target cells, e.g. airway cells, the guide RNA and Cas9 form a complex that will specifically targeted by the guiding sequence to the miRNA gene, activating Cas9 exonuclease cleavage of the targeted DNA, resulting in a double stranded break about three nucleotides upstream of the adjacent PAM site. Subsequent non-homologous end joining (NHEJ) results in an indel mutation which disrupts the expression of the targeted gene.

In alternative implementations, the miR-200 expression inhibitor may comprises other compositions for the targeted mutagenesis of a selected miR-200 gene, for example, a zinc finger nuclease (ZNF), or transcription activator-like effector nuclease (TALEN) targeted to the selected miR-200 gene.

In another embodiment, the miR-200 expression inhibitor may comprise a nucleic acid sequence which selectively interferes with transcription or processing of the targeted miRNA such as an antisense construct, short interfering RNA (siRNA), or short hairpin (shRNA) sequence.

In a first implementation, the miR-200 expression inhibitor is targeted to miR-141, for example, human miR-141, for example, to disrupt the expression of miR-141-3p. The human miRNA gene is MIR141, as known in the art, for example, Hugo Gene Nomenclature Committee (HGNC) ID number 31528. In one embodiment, the miR-141 expression inhibitor comprises a nucleic acid sequence coding for a CRISPR Cas guide RNA, comprising a subsequence of the MIR141 gene coding or non-coding strand. In one embodiment, the guide RNA sequence comprises SEQ ID NO: 15: GGCCGGCCGACAGAGAACTA. In one embodiment, the guide RNA sequence comprises SEQ ID NO: 16: CTGTACTGGAAGATGGACCC. In one embodiment, the guide RNA sequence comprises SEQ ID NO: 17: TGTACTGGAAGATGGACCCA. The miR141 inhibitors may further comprise a subsequence or variant of a sequence selected from SEQ ID NO: 15-17, for example, a subsequence comprising 15-19 continuous or non-continuous nucleotides thereof, or variants of SEQ ID NO: 15-17, and/or for example, comprising one, two, three, or more mismatches. In alternative embodiments, the miR-200 expression inhibitor inhibits expression of miR-141-5p.

In another implementation, the miR-200 expression inhibitor is targeted to miR-200a, for example, human miR-200a, for example, to disrupt the expression of miR-200a-3p and/or miR-200A-5p. The human miR-200a gene is MIR200A, as known in the art, for example, HGNC ID number 31578. In one embodiment, the miR-200a gene inactivator comprises a nucleic acid sequence coding for a CRISPR Cas guide RNA, comprising a subsequence of the MIR200a gene coding or non-coding strand.

In another implementation, the miR-200 expression inhibitor is targeted to miR-200b, for example, human miR-200b, for example, to disrupt the expression of miR-200b-3p and/or miR-200b-5p. The human miR-200b gene is MIR200B, as known in the art, for example, HGNC ID number 31579. In one embodiment, the miR-200b expression inhibitor comprises a nucleic acid sequence coding for a CRISPR Cas guide RNA, comprising a subsequence of the MIR200a gene coding or non-coding strand.

In another implementation, the miR-200 expression inhibitor is targeted to miR-200c, for example, human miR-200c, for example, to disrupt the expression of miR-200c-3p and/or miR-200c-5p. The human miR-200c gene is MIR200C, as known in the art, for example, HGNC ID number 31580. In one embodiment, the miR-200c expression inhibitor comprises a nucleic acid sequence coding for a CRISPR Cas guide RNA, comprising a subsequence of the MIR200c gene coding or non-coding strand.

In yet another implementation, the miR-200 expression inhibitor is targeted to miR-429, for example, human miR-429. The human miR-429 gene is MIR429, as known in the art, for example, HGNC ID number 13784. In one embodiment, the miR-429 expression inhibitor comprises a nucleic acid sequence coding for a CRISPR Cas guide RNA, comprising a subsequence of the MIR429 gene coding or non-coding strand.

Dosages. The agents of the invention may be delivered in any therapeutically effective dosage, which may be determined by one of skill in the art based on the release characteristics of the selected formulation, route of administration, and pharmacokinetic properties of the administered agent. Exemplary dosages may be, for example, in the range of 1 ng to 100 mg per day, for example, 10 ng to 10 mg per day, 10 ng to 10 mg per kg body weight, or 10 ng- 10 mg per square meter body surface.

Pharmaceutical Compositions. The scope of the invention further encompasses pharmaceutical compositions. The pharmaceutical compositions of the invention will comprise one or more modulators of a miR-200 miRNA in combination with pharmaceutically acceptable excipients, carriers, diluents, release formulations and other drug delivery or drug targeting vehicles, as known in the art.

In a primary implementation, the miR-200 family modulators of the invention are delivered directly to airway cells, for example by intranasal, intratracheal, or inhalation delivery. Such delivery advantageously delivers the therapeutic compositions directly to airway cells and minimizes off-target delivery to non-airway cells.

Exemplary compositions for delivery of therapeutic oligonucleotides, such as antagomirs, may include lipid nanoparticles, such as cationic lipids, phosphatidylcholine, cholesterol, and PEG. Polymer based delivery systems may include: chitosan and chitosan derivatives, such as piperazine substituted chitosans; complexes with polyethyleneimine; hyperbranched poly(beta amino esters); disulfide-constrained cyclic amphipathic peptides, PEGylated surfactant protein B and mimics thereof, such as KL4 peptide; PLGA nanoparticles coated with lipidoid; liposomes, for example, comprising DOTMA, DOPE, or mixtures thereof; methacrylate-based polymer conjugated with melittin peptide; poloxamine nanoparticles; and other compositions, for example, as reviewed in Chow et al., 2020. Inhaled RNA Therapy: From Promise to Reality, Trends in Pharmaceutical Science, 41:715-729.

Additional exemplary technologies for the delivery of inhaled agents are described, for example, in U.S. Pat. No. 9,511,198, Dry powder inhaler and system for drug delivery, by Smutney et al.; U.S. Pat. Application Publication No. 20030092666, Compositions and methods for nucleic acid delivery to the lung, by Eljamal et al.; U.S. Pat. Application Publication No. US200150224197, Inhalation Compositions, by Cifter et al.; and U.S. Pat. No. 9,554,993, Pulmonary delivery particles comprising an active agent, by Tarara et al.

In alternative implementations, the therapeutic compositions of the invention are delivered by other than inhaled delivery, for example, being formulated for administration intravenous, intra-arterial, intraperitoneal, intrapulmonary, oral, intravesicular, intramuscular, subcutaneous, transmucosal, and transdermal delivery. In one embodiment, the compositions comprises nanoparticles containing or functionalized with the selected active agent of the composition, for delivery by nanoparticle-based delivery methods. In one embodiment, the composition comprises the selected therapeutic agent admixed with a polymeric material for timed release elution of the agent. In one embodiment, the composition of the invention is coated onto an implant or drug eluting device.

In the case of miR-200 expression inhibitors, the delivery may be by any means of delivering nucleic acid sequences or expression vectors, for example, by viral vector (e.g. adenovirus or adeno-associated virus, lentivirus), nanoparticle mediated gene delivery (e.g. dendrimers, lipids, chitosan gene delivery particles, etc.), electroporation, biolistic delivery systems, microinjection, ultrasound, hydrodynamic delivery, liposomal delivery, extracellular vesicle-mediated delivery (e.g. exosome, nanovesicle), polymeric or protein-based cationic agents (e.g. polyethylene imine, polylysine), intraject systems, and DNA-delivery dendrimers.

The scope of the invention further encompasses devices for the delivery of the pharmaceutical compositions to the airway cells of the subject. Such devices will comprise an apparatus which holds the selected pharmaceutical composition and further comprising components capable of delivering a controlled dosage of such pharmaceutical composition to the airway tissues of the subject. The delivery may be accomplished by pumps, vaporizing elements such as heaters or vibrational energy sources, or by the use of compressed gases and propellants, as known in the art. In one embodiment, the device comprises a dry powder inhaler. In one embodiment, the device comprises a metered-dose inhaler. In one embodiment, the device comprises a nebulizer.

Uses and Methods of the Invention. In one aspect, the scope of the invention encompasses an inhibitor of miR-141 for use in method of treating an airway condition. In various embodiments, the airway condition may be one or more of impaired airway function, mucus overproduction in airway cells, formation of mucus plugs, defective airway epithelial function, asthma, including Th2 high asthma or fatal asthma, allergies, airway inflammation, airway hyperresponsiveness, lung disease, chronic obstructive pulmonary disease, cystic fibrosis, pathological IL-13 mediated processes, epithelial goblet cell metaplasia, mucin glycoprotein MUC5AC overproduction, chronic cough, and chronic sinus disease. In one embodiment, the inhibitor is an inhibitor of miR-141-3p. In one embodiment, the inhibitor of miR-141-3p is an inhibitor of hsa-miR-141-3p. In one embodiment, the inhibitor of miR-141 is an antagomir. In one embodiment, the antagomir comprises SEQ ID NO: 10, a subsequence thereof, or a variant thereof comprising at least 95% sequence identity to SEQ ID NO: 10. In one embodiment the inhibitor of miR-141 comprises an MIR141 expression inhibitor. In one embodiment, the MIR141 expression inhibitor comprises a nucleic acid sequence coding for a CRISPR Cas9 or like system guide RNA comprising a subsequence of the MIR141 gene coding or non-coding strand, for example, SEQ ID NO: 23 or complementary strand thereof. In various embodiments, the guide RNA sequence may comprise SEQ ID NO: 15, SEQ ID NO: 16, or SEQ ID NO: 17, or a subsequence or variant thereof.

In a related aspect, the scope of the invention encompasses a method of treating an airway condition in a subject in need of treatment therefor, comprising, administering to the subject a therapeutically effective amount of an inhibitor of miR-141. In one embodiment, the inhibitor is an inhibitor of miR-141-3p. In one embodiment, the inhibitor is an inhibitor of hsa-miR-141-3p. In one embodiment, the inhibitor is an antagomir. In one embodiment, the antagomir comprises SEQ ID NO: 10, a subsequence thereof, or a variant thereof comprising at least 95% sequence identity to SEQ ID NO: 10. In one embodiment, the administration is by inhalation, intranasal delivery, or intratracheal delivery.

In a related aspect, the scope of the invention encompasses a method of utilizing an inhibitor of miR-141 in a method of manufacturing a medicament, for example, a pharmaceutical composition as described herein. In one embodiment the scope of the invention encompasses a delivery device for the administration of the inhibitor of miR-141 wherein the delivery device holds a pharmaceutical composition comprising the inhibitor of miR-141 in combination with components for the dispensing of the inhibitor. In one embodiment, the delivery device comprises a dry powder inhaler.

In other implementations, the scope of the invention encompasses modulators of one or more miR-200 miRNAs for use in a method of treating an airway condition. The modulator may comprise a modulator of one or more miR-200 miRNAs selected from the group consisting of miR-141, miR-200a, miR-200b, miR-200c, and miR-429. The modulator may comprise an inhibitor. The modulator may comprise an inhibitor of one or more miR-200 miRNAs selected from the group consisting of miRNA-141, miR-200a-3p, miR-200b-3p, miR-200c-3p, and miR-429-3p. In some implementations, the inhibitor is an antagomir. In some embodiments, the antagomir is selected from SEQ ID NO: 11; SEQ ID NO: 12; SEQ ID NO: 13; and SEQ ID NO: 14, or a subsequence or variant of the foregoing. In some embodiments, the inhibitor is an expression inhibitor of one or more genes selected from MIR200A, MIR200B, MIC200C, and MIR429. In some embodiments, the expression inhibitor is a nucleic acid sequence coding for a CRISPR Cas9 or like system guide RNA comprising a subsequence of the MIR200A, MIR200B, MIC200C, and MIR429 gene coding or non-coding strand.

In a related aspect, the scope of the invention encompasses a method of treating an airway condition in a subject in need of treatment therefor, comprising, administering to the subject a therapeutically effective amount of an inhibitor of one or more of miR-200a, miR-200b, miR-200c, and/or miR-429. In one embodiment, the inhibitor is an inhibitor of miR-200a-3p, miR-200b-3p, and/or miR-200c-3p. In one embodiment, the inhibitor is an inhibitor of miR-200a-5p, miR-200b-5p, and/or miR-200c-5p. In one embodiment, the inhibitor is an antagomir. In one embodiment, the antagomir comprises SEQ ID NO: 11; SEQ ID NO: 12; SEQ ID NO: 13; or SEQ ID NO: 14, a subsequence thereof, or a variant thereof comprising at least 95% sequence identity to the enumerated sequence. In one embodiment, the administration is by inhalation, intranasal delivery, or intratracheal delivery.

In a related aspect, the scope of the invention encompasses a method of utilizing an inhibitor of miR-200a, miR-200b, miR200c, and/or miR-429 in a method of manufacturing a medicament, for example, a pharmaceutical composition as described herein. In one embodiment the scope of the invention encompasses a delivery device for the administration of the inhibitor of miR-200a, miR-200b, miR200c, and/or miR-429, wherein the delivery device holds a pharmaceutical composition comprising the selected inhibitor in combination with components for dispensing the inhibitor. In one embodiment, the delivery device comprises a dry powder inhaler.

### EXAMPLES

### Example 1. Epithelial miR-141 Regulates IL-13-Induced Airway Mucus Production

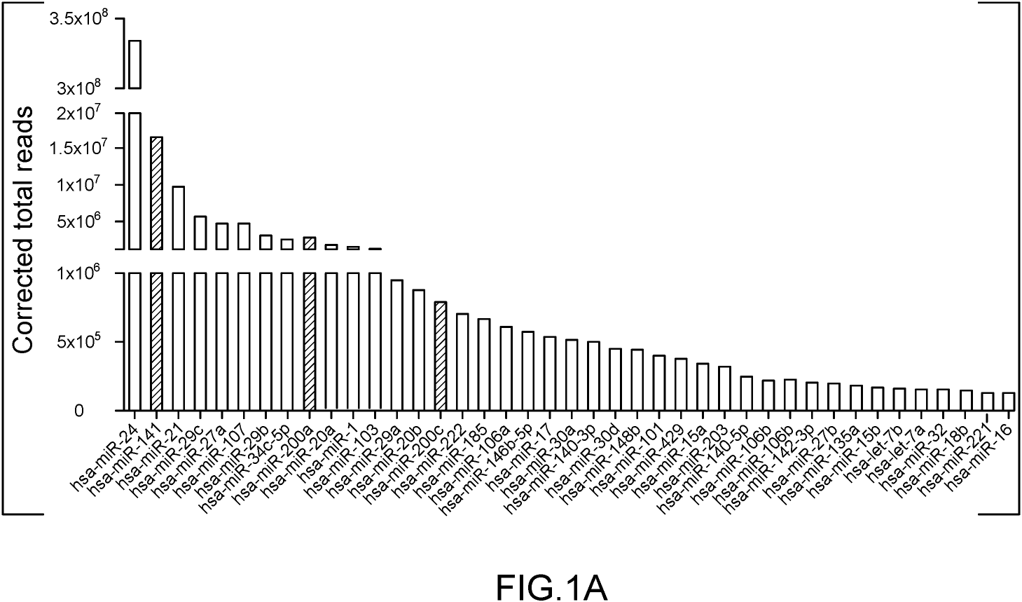

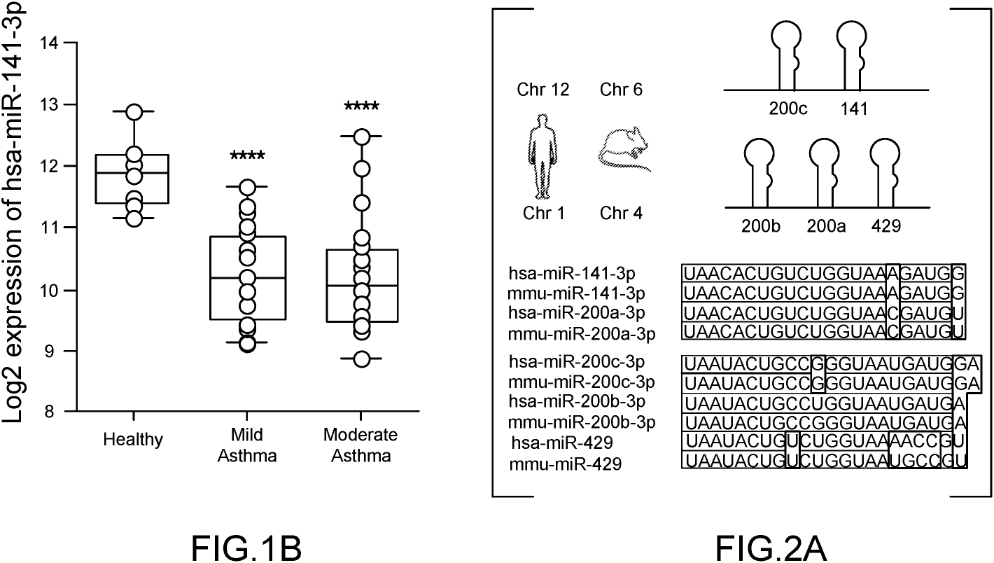

Results. miR-141 is highly expressed in human airway epithelial cells and dysregulated in asthma miRNA profiling was performed by small RNA sequencing (RNA-seq) of human bronchial epithelial brushings and found that hsa-miR-141-3p was the second most highly expressed miRNA in the airway epithelium (FIG. 1A). Including hsa-miR-141-3p, four members of the miR-141/200 family (miR-141/200a/200c/429) were among the top 40 most abundantly expressed miRNAs in bronchial epithelial brushings. Decreased hsa-miR-141-3p expression has been reported previously in bronchial epithelial brushings from 16 mild asthmatic subjects (not using inhaled corticosteroids) and 12 healthy controls using miRNA microarray data (Solberg, 2012). In additional analyses, it was found that hsa-miR-141-3p is repressed in moderate asthmatic subjects (using inhaled corticosteroids) as well (FIG. 1B). These results show that the miR- 141/200 family, including hsa-miR-141-3p, is highly expressed in human airway epithelium ex vivo, and that miR-141 is modulated in the airway epithelium in asthma.

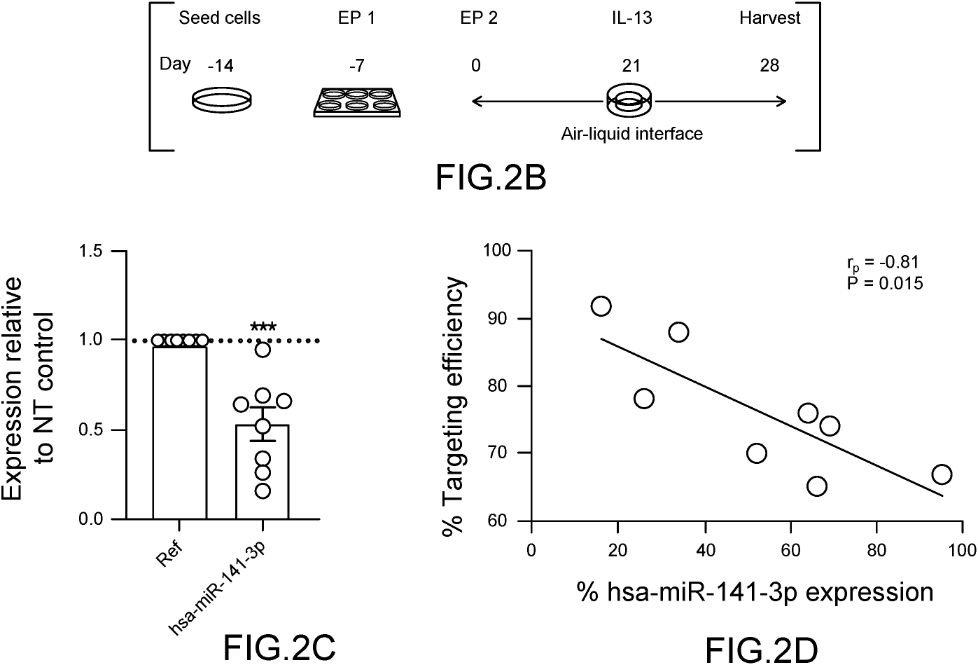

CRISPR/Cas9-targeting of the MIR141 gene successfully decreases mature hsa-miR-141-3p expression in primary human bronchial epithelial cells To study the role of miR-141 in the airway epithelium, an electroporation-based dual guide RNA (gRNA; cRNA:tracrRNA) CRISPR protocol was developed that enabled MIR141 gene repression in HBECs grown in monolayer cultures. All five family members of the miR- 141/200 family are shown in FIG. 2A. Subsequent transfer to air-liquid-interface (ALI) generated a fully differentiated airway epithelium (timeline outlined in FIG. 2B). On day 28, HBECs that received either MIR141 gene-targeting gRNA or non-targeting (NT) gRNA control were harvested and DNA was isolated to confirm editing efficiency by Sanger sequencing. Across 9 unique HBEC donors, targeting efficiency was estimated for MIR141 knockdown to be 65-95%. The expression of mature hsa-miR-141-3p was significantly reduced upon MIR141-targeting compared to the NT control (FIG. 2C). Expression of other miR-141/200 family miRNAs in MIR141-targeted HBECs were not significantly reduced compared to NT control. Furthermore, repression of hsa-miR-141-3p expression in CRISPR/Cas9-targeted cells correlated significantly with the estimated targeting efficiency (FIG. 2D).

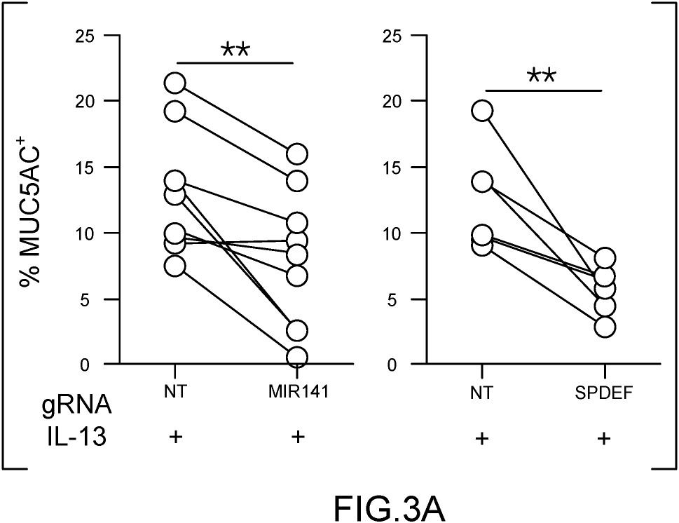

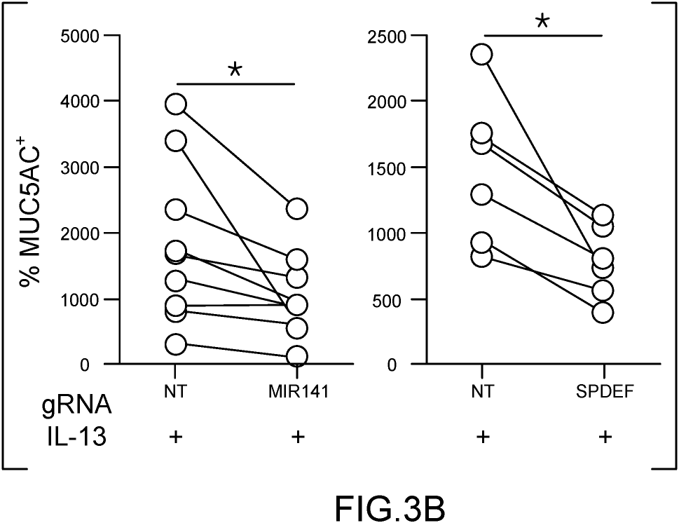

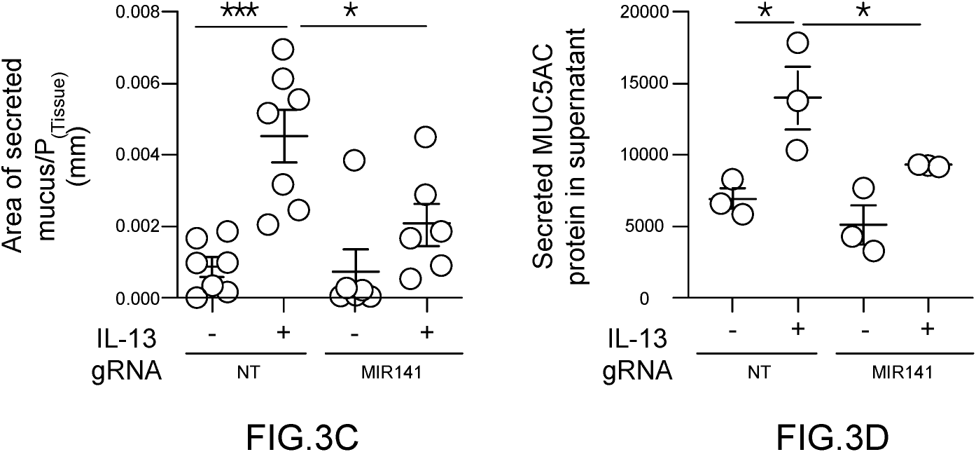

miR-141 repression reduces IL-13-induced mucus in primary HBECs The effect on the IL-13-inducible airway mucin MUC5AC following MIR141 gene-editing was studied in primary HBECs. Using intracellular flow cytometry, it was found that MIR141-targeting significantly decreased the frequency of MUC5AC-expressing cells following IL-13 stimulation compared to NT gRNA control HBEC cultures (FIGS. 3A-B). CRISPR/Cas9- targeting of the goblet cell transcription factor SPDEF also resulted in significantly decreased MUC5AC⁺ cells, as recently shown. Gene-editing reduced both the frequency of MUC5AC-expressing cells and mean fluorescence intensity (MFI), reflecting the amount of MUC5AC-binding antibodies, in MIR141 and SPDEF-targeted HBECs compared to HBECs that received the NT gRNA control (FIGS. 3C-D). Decreased MUC5AC expression was also apparent in immunofluorescent staining of MIR141-targeted IL-13-stimulated HBECs in filter sections. MUC5B was weakly detected in both the NT gRNA control and MIR141-targeted cells. Image analysis of Alcian Blue-Periodic Acid Schiff (AB-PAS)- stained filters revealed a significant reduction of the area of secreted mucus in MIR141- targeted HBECs compared to NT control HBECs under IL-13-stimulated conditions (FIG. 3E). In addition, secreted MUC5AC protein was quantified by dot blot analysis of apical wash samples collected from IL-13-stimulated or untreated ALI cultures (FIG. 3F). The results confirmed a significant decrease of MUC5AC following MIR141 gene-editing when compared to NT gRNA controls. These results indicate that miR-141 regulates IL-13-induced MUC5AC production by epithelial cells.

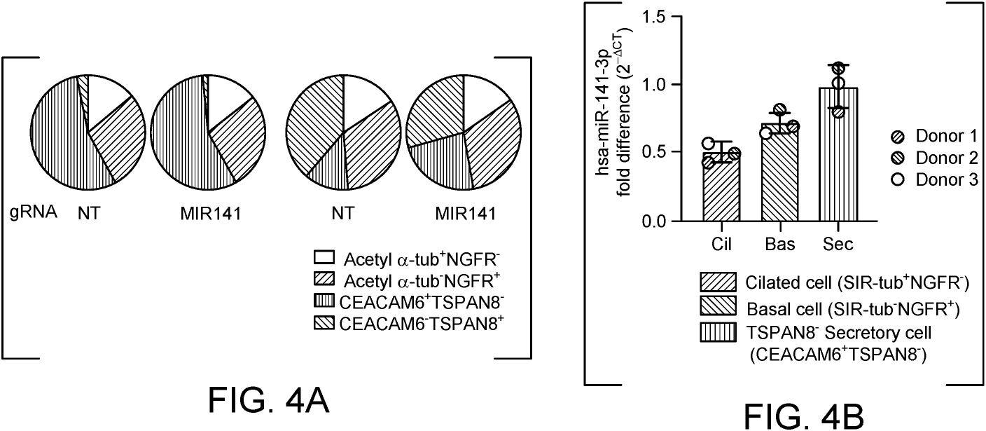

Epithelial miR-141 repression is associated with a reduction in mucus-producing goblet cell numbers To investigate the mechanism by which miR-141 regulates MUC5AC, specific changes in airway epithelial subpopulations in response to MIR141-targeting were measured by flow cytometry. Airway epithelial subpopulations were analyzed using a panel of antibodies targeting subset-specific cellular markers that have been described previously and newly identified by single cell RNA-seq (scRNA-seq) analysis of human bronchial brushings. MIR141 gene-editing in IL-13-stimulated HBECs resulted in significantly lower frequency of TSPAN8⁺ secretory cells (defined as acetylated α-tubulin⁻ NGFR⁻CEACAM6⁺TSPAN8⁺) compared to IL-13-stimulated NT gRNA control HBECs. TSPAN8⁺ secretory cells were only present in IL-13-stimulated cultures and was the major MUC5AC-producing population detected by intracellular MUC5AC- staining. The baseline cellular composition was similar in MIR141-targeted and NT gRNA control HBEC cultures under untreated conditions (FIG. 4A). Using non-permeabilized cells, fresh IL-13-stimuled ALI-cultured HBECs from 3 unique donors were FACS-purified to enable analysis of subset-specific expression of miR-141. It was found that hsa-miR-141-3p was enriched in TSPAN8⁺ secretory cells (SiR-tubulin⁻NGFR⁻CEACAM6⁺) compared to ciliated cells (SiR-tubulin⁺NGFR⁻) and basal cells (SiR-tubulin⁻NGFR⁺) (FIG. 4B). Additionally, the expression level of hsa-miR-141-3p was similar in TSPAN8⁺ and TSPAN8⁻ secretory cells, where TSPAN8⁻ cells are likely to be precursors of TSPAN8⁺ secretory cells. These results suggest that a reduced level of miR-141 in CRISPR/Cas9-targeted cultures affects secretory/goblet cells. Further analysis revealed that the increased number of TSPAN8⁺ secretory cells and MUC5AC⁺ goblet cells (defined as acetylated α-tubulin⁻ NGFR⁻ CEACAM6⁺TSPAN8⁺MUC5AC⁺) significantly correlated with hsa-miR-141-3p expression levels in IL-13-stimulated HBEC cultures, consistent with the finding that hsa-miR-141-3p is enriched in FACS-sorted secretory cells. Ciliated cells (acetylated α- tubulin ⁺NGFR⁻) and basal cells (acetylated α-tubulin⁻NGFR⁺) displayed no significant modulation in relation to hsa-miR-141-3p expression, suggesting that miR-141 targets genes that specifically regulate secretory/goblet cells. Moreover, analysis of other miR-141/200 family miRNAs revealed significant correlations between increasing frequency of TSPAN8⁺ secretory cells in IL-13-stimulated HBECs and expression levels of hsa-miR-200b-3p, hsa-miR-200c-3p and hsa-miR-429. No other airway epithelial subpopulations correlated with the expression of miR-200b/c/429, including MUC5AC⁺ goblet cells, which only demonstrated a significant association to hsa-miR-141-3p expression.

MIR141-targeting of epithelial cells interferes with the response to IL-13 and results in reduced expression of goblet cell genes To study the consequences of miR-141 repression in epithelial cells on a transcriptional level, MIR141-targeted HBECs and NT gRNA control HBECs were analyzed by RNA-seq. A goblet cell gene signature was generated by scRNA-seq analysis of bronchial epithelial brushings from 4 allergic asthmatic individuals that were collected 24 h following segmental allergen challenge or diluent control. Cellular clusters in epithelial brushings after allergen challenge demonstrated a large overlap with diluent control samples and a total of 18 distinct cellular clusters were defined. The cluster analysis identified a signature of 100 genes ( that was significantly enriched in goblet cells and included well-known goblet cell genes such as the mucins MUC5AC, MUC5B, MUC1, and SCGB1A1 (Secretoglobin Family 1A Member 1), TFF3 (Trefoil Factor 3), CEACAM6 (CEA Cell Adhesion Molecule 6) and SPDEF. The 100-goblet cell gene signature was analyzed across 4 NT control HBEC donors and 4 MIR141-targeted HBEC donors using Gene Set Enrichment Analysis (GSEA). In bulk RNA-seq analysis, this goblet cell gene signature was highly enriched in the IL-13-stimulated NT gRNA control condition compared to IL-13-stimulated MIR141-targeted cells, supporting the previous findings of decreased goblet cell frequency in MIR141-targeted HBECs by flow cytometry. IL-13 stimulated NT gRNA control HBECs also exhibited a significant enrichment of ciliated cell genes, however, the goblet cell gene signature displayed the highest enrichment score. Furthermore, analysis of IL-13-induced changes in global gene expression using Ingenuity Pathway Analysis (IPA) identified the goblet cell transcription factor SPDEF to be the most likely upstream regulator of the transcriptional changes induced by IL-13 in NT gRNA control HBECs. The SPDEF-network had activation z-score of 3.96 and demonstrated a significant overlap with the RNA-seq data set (P-value = 2.5x10⁻¹²) where the expression of 21 of 27 genes downstream of SPDEF was consistent with activation of SPDEF. In contrast, IPA analysis of differentially expressed genes in IL-13 stimulated MIR141-targeted cells revealed a complete lack of goblet cell-related networks. Indeed, in response to IL-13 stimulation, a significant number of genes were differentially expressed in NT gRNA control HBECs (both upregulated and downregulated) but not in MIR141-targeted HBECs, which may suggest that miR-141 expression is required for a normal epithelial response to IL-13 and goblet cell development.

miR-141 repression leads to increased basal cell gene expression Basal cells of the airway epithelium give rise to multiple cell lineages, including mucus producing goblet cells. To learn more about how miR-141 repression interferes with responses of mucus secretory cells, the basal cell gene signatures obtained from scRNA-seq of bronchial brushings were included in the GSEA analysis of MIR141-targeted and NT gRNA control HBECs. It was found that MIR141-targeted HBECs exhibited a significant enrichment of basal cell genes compared to NT gRNA controls. Next, the expression of miR-141 was studied, as assessed by miRNA sequencing, in differentiating ALI-cultures every 2-3 days from airlifted confluent cultures on day 4 to a fully differentiated epithelium on day 22. Hsa-miR-141-3p exhibited a dynamic expression pattern with the lowest expression day 4 and stepwise increases reaching a peak on day 22. These findings suggested that MIR141 may be involved in the transition of basal cells into mucus secretory goblet cells that occurs in human trachea and ALI cultures.

Large numbers of predicted and confirmed miR-141 targets are expressed during basal- to-mucus secretory cell transition The basal and goblet cell signatures derived from scRNA-seq were compared with 7 distinct transitional states from basal-like cells to fully competent MUC5AC-expressing mucus secretory cells that were previously defined by pseudotime gene cluster analysis of an independent data set, as described in Goldfarbmuren KC, et al. Dissecting the cellular specificity of smoking effects and reconstructing lineages in the human airway epithelium. bioRxiv; 2019 Apr. Available from: http://biorxiv.org/lookup/doi/10.1101/612747. The genes defining these transitional states corresponded well with the basal and goblet cell signatures. Using TargetScan v7.2 the frequency of genes was analyzed in the 7 transitional gene clusters with a predicted conserved hsa-miR-141-3p seedmatch in the 3′UTR. A large number of predicted miR-141 targets (126 genes, 14% of all hsa-miR-141-3p predicted targets) overlapped with genes across the 7 clusters. The highest concentration of predicted targets was found in the early transitional ‘Intermediate 1’ cluster. The largest defined groups of the 126 predicted target genes encoded enzymes and transcriptional regulators. To increase the confidence of miR-141 targets identified during basal-to-secretory cell differentiation, a recently published CLEAR-CLIP data set was mined that captured individual miRNAs and their targeted RNA sites in wild-type, miR-200 family induced and miR-200 family deficient murine epithelial cells, as described in Bjerke GA, Yi R. Integrated analysis of directly captured microRNA targets reveals the impact of microRNAs on mammalian transcriptome. RNA. 2020 Mar;26(3):306-23. Almost all 126 genes had a miR-141-3p binding site that was conserved in the murine genome. Using differential CLEAR-CLIP peaks, 38 genes were confirmed to be experimentally captured in wild-type or miR-200-induced epithelial cells but not in miR-200 family deficient cells. Differential RNA-seq analysis of the 38 experimentally confirmed target genes in IL-13-stimulated MIR141-targeted and NT control HBECs revealed a significant number of derepressed miR-141-3p targets in MIR141 gene-edited HBECs, indicating that miR-141 may regulate a network of genes that are repressed during normal goblet cell differentiation. Some miR-141 target genes that were derepressed in MIR141-targeted HBECs were broadly detected by scRNA-seq in goblet, basal and ciliated cells from asthmatic airways In addition, several miR-141 target genes were selectively expressed in goblet, basal or ciliated cells, and some genes were differentially expressed following segmental allergen challenge compared with diluent controls. For instance, allergen challenge induced PDCD4 (Programmed cell death 4) expression in goblet cells. Depletion of PDCD4 by RNA interference in vivo via intranasal administration suppresses airway mucus secretion in OVA-induced allergic airway inflammation.

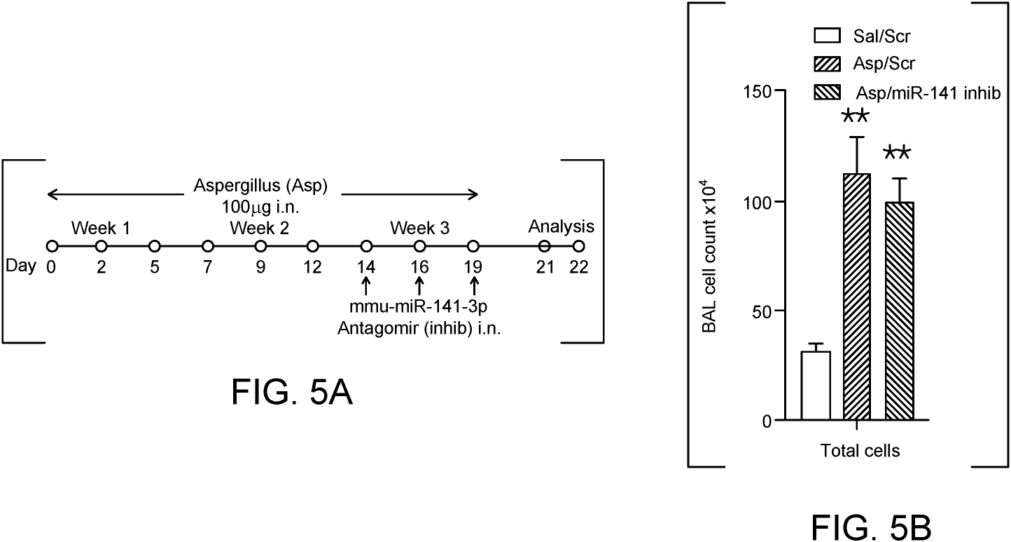

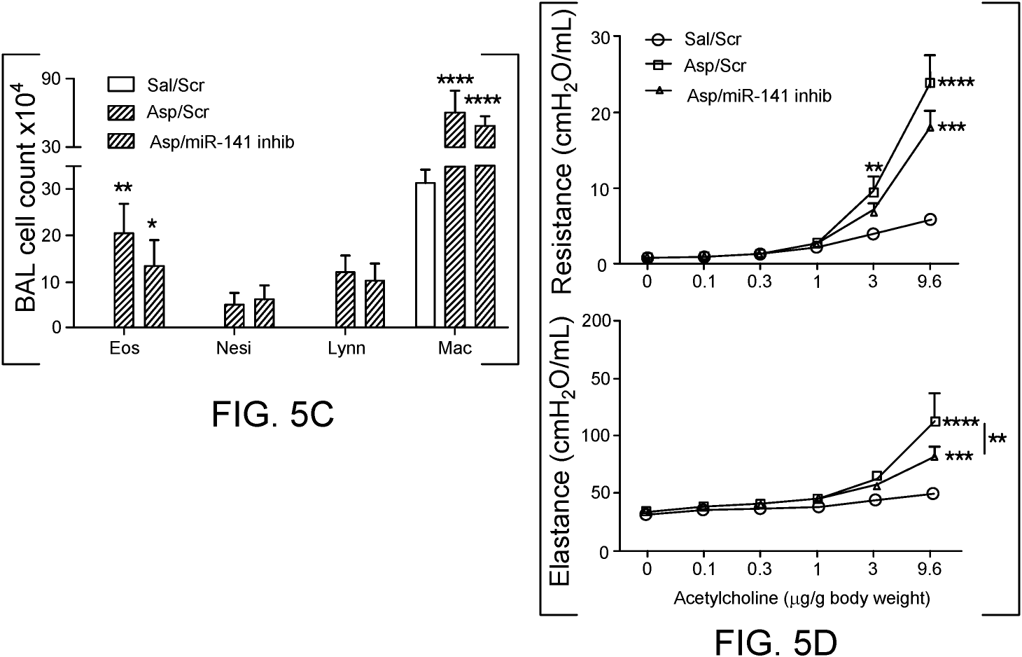

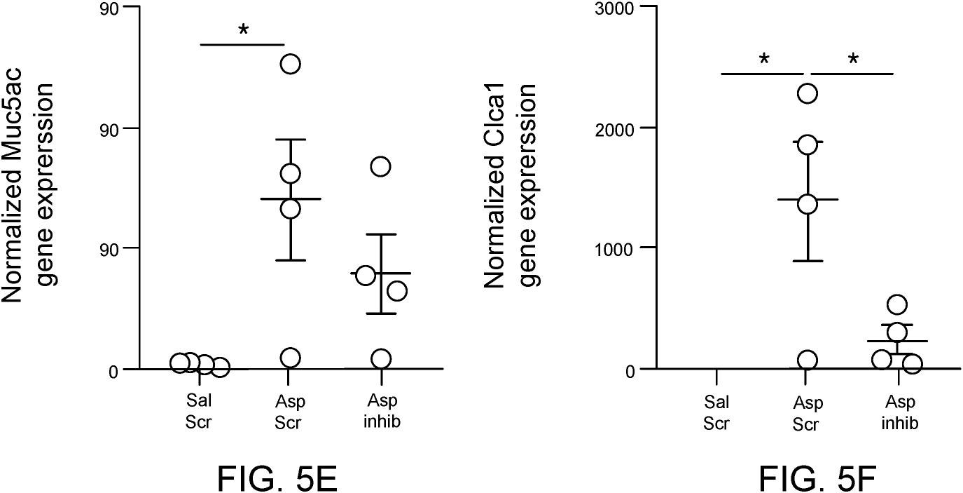

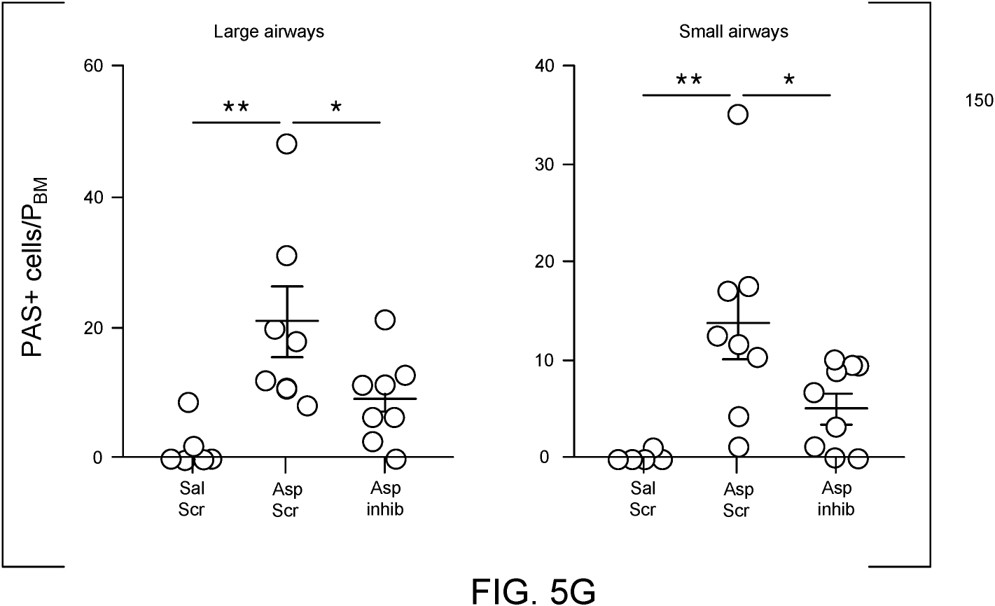

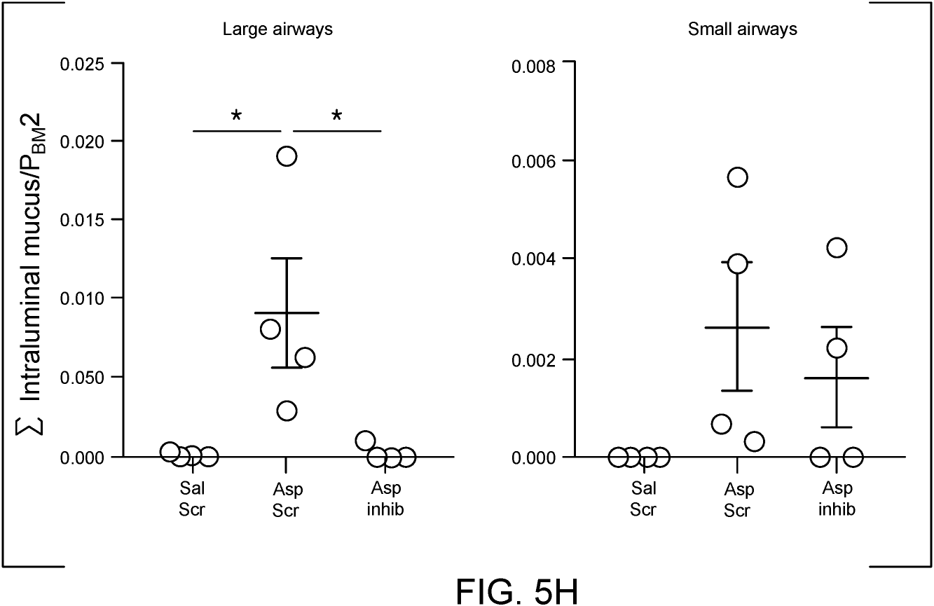

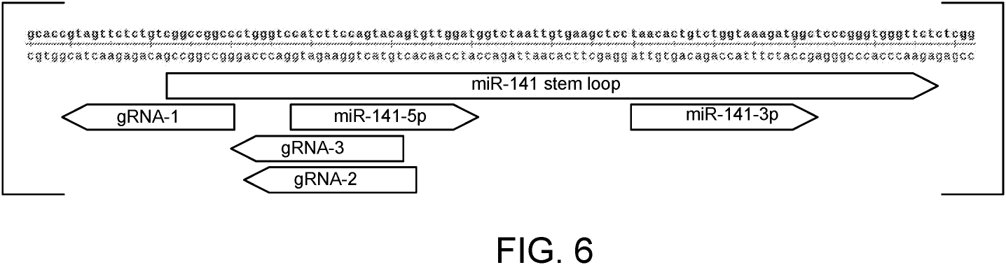

Inhibition of mmu-miR-141-3p in vivo improves allergen-induced airway hyper-responsiveness Previous studies in mice have demonstrated highly efficient epithelial uptake of antagomirs administered directly to the airways, for example, as described in Zhong B, et al. Pdcd4 modulates markers of macrophage alternative activation and airway remodeling in antigen-induced pulmonary inflammation. J Leukoc Biol. 2014;96(6): 1065-75. To study the effects of miR-141 inhibition on mucus regulation in vivo, a model of allergic asthma was used in which mice were challenged intranasally with the fungal allergen Aspergillus fumigatus (FIG. 5A), or administered sterile saline as a sham challenge control. Analysis of cellular populations in bronchoalveolar lavage (BAL) revealed that the composition of inflammatory cells was unaffected by mmu-miR-141-3p antagomir treatment (FIGS. 5B-C). Airway hyper-responsiveness was measured 48-72 hours after the final allergen challenge and it was found that Aspergillus-challenged mice that received the mmu-miR-141-3p antagomir were less reactive to acetylcholine compared to Aspergillus-challenged mice that received the scrambled antagomir as assessed by total respiratory system resistance and elastance (FIG. 5D). Furthermore, MUC5AC gene expression in whole lung homogenate displayed a decreasing trend in mice that received mmu-miR-141-3p antagomir (FIG. 5E), and the goblet cell-specific gene CLCA1 was significantly downregulated upon mmu-miR-141-3p antagomir treatment in allergen- challenged lungs (FIG. 5F), demonstrating that mmu-miR-141-3p inhibition blocked allergen-induced goblet cell differentiation.

Inhibition of mmu-miR-141-3p decreases epithelial mucus induction in vivo Lung sections from Aspergillus-challenged mice were prepared to examine mucus-producing cells in the lung tissue. Mice that were challenged with airway allergen and which received mmu-miR-141-3p antagomir had significantly decreased numbers of mucus-expressing epithelial cells in the large and small airways (FIG. 5G). Furthermore, large airways also exhibited decreased secreted mucus in the airway lumen (FIG. 5H) after mmu-miR-141-3p antagomir treatment compared to scramble antagomir treatment, demonstrating that inhibition of mmu-miR-141-3p has a therapeutic effect in reducing key features of allergen-induced asthma.

Discussion. Although the study of miRNAs in asthma and allergic inflammation is a relatively young field, it is evident that these conditions are accompanied by changes in miRNA expression, which, in turn, promote disease pathology. Yet, a tremendous amount of work remains to assign physiological functions to individual miRNAs and to integrate them in the understanding of cellular mechanisms in specific pathological contexts. In the current study, it was found that miR-141 regulates the increase in airway epithelial goblet cell numbers, goblet cell MUC5AC gene and protein expression and epithelial mucus production that occur after stimulation with IL-13. MUC5AC is a major component of the pathological mucus gel in T2-high asthma, a condition associated with increased IL-13 and mucus plugging is a feature of both severe chronic T2-high asthma and of severe asthma exacerbations. To determine whether inhibition of miR-141 could represent a therapeutic strategy in asthma, it was demonstrated herein that intranasal administration of an antagomir specific to mmu-miR-141-3p reduces airway hyper-responsiveness and mucus production without altering cellular inflammation, showing a direct effect on epithelial cells and not by indirect inflammatory cell signaling. These data demonstrate that inhibition of miR-141 provides a novel strategy for treatment of pathological mucus production and airflow obstruction in T2-high asthma. Although the in vitro and in vivo data indicate that miR-141 promotes pathological mucus production in the setting of T2 inflammation, epithelial miR-141 may be downregulated in the airways of mild-moderate asthmatics where mucus metaplasia is a central feature. In the current study, it was found that MIR141-targeting of IL-13-stimulated HBECs increased basal cell gene expression. Multiple experimentally confirmed miR-141 targets were confirmed in a gene network expressed early during basal-to-mucus secretory cell differentiation, many of which were derepressed in MIR141-targeted cells and differentially expressed in asthmatic airways following segmental allergen challenge. The initially low expression of miR-141 in differentiating ALI cultures would allow expression of basal cell genes that are targeted by miR-141, whereas subsequent increase of miR-141 in differentiating cells would suppress these genes to promote development of mucus secretory cells. Indeed, it was found that miR-141 expression was enriched in FACS-sorted secretory cells compared to basal and ciliated cells, and the increased expression level of miR-141 correlated specifically with the frequency of goblet cell populations, but not with non-mucus producing airway epithelial subpopulations in IL-13-stimulated HBEC cultures. The date indicate that that long-term exposure to IL-13 and other inflammatory mediators that are present in the asthmatic milieu of the airways promote downregulation of miR-141which contributes to defective epithelial responses.

The miR-141/200 family is encoded in two genomic clusters, which give rise to five highly homologous mature miRNAs that are well-conserved across species. A recent study investigating CRISPR/Cas9-based editing of miRNA clusters found that a mutation in one hairpin could affect the expression of a miRNA that resided in the other hairpin of the same cluster. This observation was thought to be due to changes in the tertiary structure of the pri-miRNA leading to a differential expression of the mature miRNA. However, the authors also reported that mutations were well-tolerated, provided they did not disrupt critical elements such as stem length, bulge position and terminal loops (39). These findings are important since they imply that CRISPR/Cas9-editing of miRNAs can affect processing of the hairpin in a dual manner; directly through sequence alteration and disruption of sequence motifs, or structurally through changes to the pri-miRNA, thus highlighting the complexity of CRISPR/Cas9-targeting of miRNAs. Herein, miR-141 significantly downregulated hsa-miR-141-3p expression with relative preservation of the expression of the other miR-141/200 family miRNAs. Importantly, miR-141 has an identical seed sequence to miR-200a such that one could expect miR-141 and miR-200a to play additive or synergistic roles in airway epithelial responses. However, the results clearly show that targeting miR-141 alone is sufficient to repress IL-13-induced mucus production. The remaining miR-141/200 family members are less homologous to miR-141 with additional overlapping effects. Indeed, a recent study of the miR141/200 family found that the two subgroups (distinguished by the seed sequence; 141/200a and 200b/200c/429) bound to largely distinct sites and cross-seed recognition was rare (i.e. 141/200a binding of RNAs with a 200b/200c/429 seedmatch). However, many genes were regulated by multiple family members sharing the same seed sequence suggesting that the miR-141/200 family cooperates in target recognition. Interestingly though, in the study, hsa-miR-200a-3p was the only miR-141/200 family member whose expression did not show any relationship to the frequency of goblet cells in IL-13-stimulated HBEC cultures. This suggests that miR-141 and miR-200a may not share functional overlap in mucus regulation, or that the lower expressed miR-200a alone may not be sufficient to compensate for reduced expression of miR-141.

In the present disclosure, the list of reported transcriptional regulators and other factors that are targeted by miR-141 is expanded and expressed during mucus secretory cell development. The functional diversity of epithelial tissues is dictated by the composition of differentiated cell subsets. Here it demonstrated that targeting MIR141 altered airway epithelial subpopulations, especially in IL-13-stimulated conditions. IL-13 stimulation of ALI-cultured HBECs potently promotes mucus metaplasia and several recent studies have provided a detailed view of the hierarchical lineage of airway epithelial cells. Basal cells also give rise to club cells that can, in turn, transdifferentiate into ciliated cells and goblet cells, and asthmatic lungs contain ciliated cells that co-express a number of goblet genes including MUC5AC. In the present disclosure, it was found that MIR141-targeting resulted in a significant difference in TSPAN8⁺ secretory cell frequency by flow cytometry, but did not alter frequencies of ciliated and basal cells. The specific repression of mucus secretory cells upon MIR141-targeting can explain the reduction in secreted and intracellular mucus that was observed both in the in vitro system and in vivo model of allergic asthma. An inhaled antagomir-based therapeutic approach is attractive in this context for several reasons. miR-141 expression is largely restricted to the bronchial tree in mice and therefore accessible to intranasal antagomir administration. Previous studies in mice have reported close to 100% uptake in the airway epithelium using intranasal delivery. Indeed, mmu-miR-141-3p inhibition in the present study resulted in CLCA1 gene downregulation, which suggests defective goblet cell differentiation in response to allergen challenges to the airway. This result highlights that modulation of miR-141 in the airways has important beneficial effects in asthma.

In summary, the studies of airway epithelial mucus regulation disclosed herein identified a miRNA that is differentially expressed in asthma, and that upon repression, downregulates epithelial mucus production in vivo and in vitro and reduces airway hyper-responsiveness. Given the pathogenic role that mucus overproduction and plugging plays in airflow obstruction in chronic severe asthma and in severe asthma exacerbations, and the lack of therapeutics that specifically target mucus production in the airway, miR-141 and/or its mRNA targets are valuable new therapeutic targets in T2-high asthma.

Methods. Cell culture conditions Culture plates and inserts were precoated with human placental collagen (15 µg/cm²) (1). Human bronchial epithelial cells (HBECs) were seeded in medium that consisted of a 3:1 ratio of F12 and DMEM and was supplemented with 5% heat inactivated FBS, 100 U/ml penicillin, 100 µg/ml streptomycin, 10 µg/ml gentamicin, 250 ng/ml 5 µg/ml bovine insulin, 8.4 ng/ml cholera toxin, 25 ng/ml hydrocortisone and 10 ng/ml rh-EGF. Rho-associated protein kinase inhibitor Y-27632 (10 µM, ‘ROCK inhibitor’) was added right before use. Culture medium used for cells grown at air- liquid-interface (ALI) consisted of 1:1 ratio of LHC Basal Medium and DMEM supplemented with 0.5 mg/ml BSA, 0.24 mg protein/ml BPE, 5 µg/ml bovine insulin, 10 µg/ml transferrin, 0.1 µM hydrocortisone, 0.01 µM triiodothyronine, 2.7 µM epinephrine, 0.5 ng/ml rh-EGF, 0.05 µM retinoic acid, 0.5 µM phosphorylethanolamine, 0.5 µM ethanolamine, 3 µM zinc sulfate, 100 U/ml penicillin, 100 µg/ml streptomycin and 2 mM L-glutamine.

Preparation of crRNAs crRNAs were resuspended in 150 mM KCl and 10 mM Tris-HCl, pH 7.4. Ribonucleoprotein (RNP) complex was prepared by first incubating 160 µM, 1 µL of crRNA Lafayette, CO) with 160 µM, 1 µL tracrRNA, at 37° C. for 30 min yielding 80 µM gRNA. The 80 µM gRNA was then added 1:1 with 40 µM, 2 µL rCas9, recombinant Cas9 (MacroLab, Berkeley, CA), yielding 20 µM RNP, which was incubated at 37° C. for 15 min. An electroporation enhancer DNA oligonucleotide (100 µM, 1 µL) was added to the RNP to enhance efficiency of the delivery of the complex to the cells.

Preparation of HBECs for flow cytometry ALI cultured HBECs were harvested on day 28 for analysis by flow cytometry. At harvest, 10 mM DTT in PBS with Ca²⁺Mg²⁺ was added to the apical compartment and incubated for 10 min at 37° C. DTT wash was collected for dot blot analysis. Cells were washed with PBS, then incubated for a maximum of 15 min at 37° C. in 0.25% trypsin with 2.21 mM EDTA (Corning) which was added to the apical and basolateral compartments. Cell culture medium containing 5% FBS was added to neutralize the enzymatic activity and cells were washed in PBS followed by fixation in 4% PFA (ThermoFisher Scientific) for 8 min on ice. Cells were washed in PBS and finally resuspended in plain PBS and stored at -80° C. until analysis.

Secreted MUC5AC by dot blot. At harvest, the apical compartment of ALI cultured HBECs were washed with 10 mM DTT for 10 minutes at 37° C. The washes were stored at -80° C. until analysis. Dot blot was adapted from the slot blot technique described earlier (3) and as performed previously (4). Following thawing, samples were diluted and spotted on to nitrocellulose. The membrane was allowed to dry, blocked in 4% milk, then stained with an anti-MUC5AC primary antibody (MAN-5ACI). The blot was subsequently incubated with a HRP conjugated anti-rabbit secondary antibody and detected using TMB peroxidase substrate.

Histologic staining and immunofluorescence 6.5 mm ALI filters inserts were collected day 28 and placed in Carnoy’s solution (6:3:1 ratios of methanol, chloroform, glacial acetic acid) for 30 min at RT. Briefly, filters were washed in concentrated methanol (2x20 min) followed by 4-5 washes in PBS. The filters were embedded in paraffin and cut in 5 µm sections that were later stained with Hematoxylin and Eosin (H&E), AB-PAS and fluorescent antibodies. H&E staining and AB-PAS staining was performed on deparaffinized sections according to standard protocols. Sections were hydrated and AB-PAS stained sections were placed in 3% acetic acid for 3 min followed by 1% alcian blue pH 2.5 for 30 min. Sections were then washed in tap water followed by DI water. 10 min incubation in 1% periodic acid was used to oxidize the sections, they were then washed in tap water and DI water. Sections were placed in Shiff’s reagent for 20 min followed by 10 min wash in tap water and 30 sec incubation in Mayer’s Hematoxylin. 1 min wash in tap water was followed by 10 sec incubation in lithium carbonate. Sections were dehydrated according to standard protocol and mounted using Immunofluorescence staining using primary antibodies mouse monoclonal anti-MUC5AC (1:200 dilution) and rabbit polyclonal anti-MUC5B (1:200 dilution). After washing, slides where incubated with secondary antibodies Alexa Fluor(TM) 488 goat anti-mouse and Alexa Fluor(TM) 647 goat anti-rabbit (at 1:200 dilution for 2 h, Jackson ImmunoResearch Laboratories, West Grove, PA) DAPI was used to stain nuclei.

Periodic-Acid Schiff (PAS) staining Lung tissue from allergen-challenged mice treated with mmu-miR-141-3p antagomir or scrambled antagomir and saline-challenged control mice was fixed in formalin and processed as previously described in Dunican et al., Autopsy and Imaging Studies of Mucus in Asthma. Lessons Learned about Disease Mechanisms and the Role of Mucus in Airflow Obstruction. Annals ATS. 2018 Nov 1;15(Supplement_3): S184-91. Assessment of PAS⁺ cells was performed on blinded lung tissue sections and analyzed using Image J Software (NIH, LOCI, University of Wisconsin). Airways with a basement perimeter (PBM) >0.80 mm were considered as central airways (‘large airways’) and a PBM <0.80 mm was considered peripheral airways (‘small airways’).

Analysis of miR-141 gene targets DIANA-microT was used to obtain genomic coordinates of mmu-miR-141-3p binding sites of target genes predicted by TargetScan v.7.2 (8 mer, 7 merM8 and 7 merA1 motifs). CLEAR-CLIP sequencing data was downloaded from the Gene Expression Omnibus (GEO) at GSE102716. Perfect overlap of genomic target sites and CLEAR-CLIP miR-141-3p peaks in wild type or miR-200 family induced epithelial cells, and absence of miR-141-3p CLEAR-CLIP peak in miR-200 family deficient cells were considered experimentally confirmed miR-141 targets.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

1-35. (canceled)

36. A method of treating an airway condition in a subject in need of treatment therefor, comprising
administering to the subject a therapeutically effective amount of an inhibitor of miR-141, miR-200a, miR-200b, miR200c, or miR429.

37. The method of claim 36, wherein
the airway condition is one or more condition selected from the group consisting of impaired airway function; mucus overproduction in airway cells; formation of mucus plugs; defective airway epithelial function; asthma; Th2 high asthma; fatal asthma; allergies; airway inflammation; airway hyperresponsiveness; lung disease; chronic obstructive pulmonary disease; cystic fibrosis; pathological IL-13 mediated processes; epithelial goblet cell metaplasia; mucin glycoprotein MUC5AC overproduction; chronic cough; and chronic sinus disease.

38. The method of claim 36, wherein
the method comprises administering to the subject a therapeutically effective amount of an inhibitor of miR-141.

39-41. (canceled)

42. The method of claim 41, wherein
the inhibitor comprises an antagomir.

43. The method of claim 42, wherein
the antagomir comprises SEQ ID NO: 10, a subsequence thereof, or a variant thereof comprising at least 95% sequence identity to SEQ ID NO: 10.

44-48. (canceled)

49. The method of claim 36, wherein
the method comprises administering to the subject a therapeutically effective amount of an inhibitor of miR-200a.

50. The method of claim 49, wherein
the inhibitor comprises an antagomir.

51. The method of claim 50, wherein
the antagomir comprises a nucleic acid sequence having at least 95% sequence identity to SEQ ID NO: 11.

52. The method of claim 36, wherein
the method comprises administering to the subject a therapeutically effective amount of an inhibitor of miR-200b.

53. The method of claim 52, wherein
the inhibitor comprises an antagomir.

54. The method of claim 53, wherein
the antagomir comprises a nucleic acid sequence having at least 95% sequence identity to SEQ ID NO: 12.

55. The method of claim 36, wherein
the method comprises administering to the subject a therapeutically effective amount of an inhibitor of miR-200c.

56. The method of claim 55, wherein
the inhibitor comprises an antagomir.

57. The method of claim 56, wherein
the antagomir comprises a nucleic acid sequence having at least 95% sequence identity to SEQ ID NO: 13.

58. The method of claim 36, wherein
the method comprises administering to the subject a therapeutically effective amount of an inhibitor of miR-429.

59. The method of claim 58, wherein
the inhibitor comprises an antagomir.

60. The method of claim 59, wherein
the antagomir comprises a nucleic acid sequence having at least 95% sequence identity to SEQ ID NO: 14.

61. An antagomir of miR-141, comprising
a nucleic acid sequence having at least 95% sequence identity to SEQ ID NO: 10.

62. An antagomir of an miR-200 family member, comprising
a nucleic acid sequence having at least 95% sequence identity to a sequence selected from SEQ ID NO: 11, SEQ ID NO: 12, SEQ ID NO: 13, or SEQ ID NO: 14.


SEQUENCE LISTING (23 sequences)

SEQ ID NO: 1 - RNA, 22 nt, Homo sapiens
  hsa-miR-141-3p mature micro-RNA
uaacacugucugguaaagaugg

SEQ ID NO: 2 - RNA, 22 nt, Homo sapiens
  hsa-miR-141-5p mature micro-RNA
caucuuccaguacaguguugga

SEQ ID NO: 3 - RNA, 22 nt, Homo sapiens
  hsa-miR-200a-3p mature micro-RNA
uaacacugucugguaacgaugu

SEQ ID NO: 4 - RNA, 22 nt, Homo sapiens
  hsa-miR-200a-5p mature micro-RNA
caucuuaccggacagugcugga

SEQ ID NO: 5 - RNA, 22 nt, Homo sapiens
  hsa-miR-200b-3p mature micro-RNA
uaauacugccugguaaugauga

SEQ ID NO: 6 - RNA, 22 nt, Homo sapiens
  hsa-miR-200b-5p mature micro-RNA
caucuuaccggacagugcugga

SEQ ID NO: 7 - RNA, 23 nt, Homo sapiens
  hsa-miR-200c-3p mature micro-RNA
uaauacugccggguaaugaugga

SEQ ID NO: 8 - RNA, 22 nt, Homo sapiens
  hsa-miR-200c-5p mature micro-RNA
cgucuuacccagcaguguuugg

SEQ ID NO: 9 - RNA, 22 nt, Homo sapiens
  miR-429 mature micro-RNA
uaauacugucugguaaaaccgu

SEQ ID NO: 10 - RNA, 22 nt, Artificial Sequence
  Description of Artificial Sequence: Synthetic antagomir to hsa-miR141-3p sequence
ccaucuuuaccagacaguguua

SEQ ID NO: 11 - RNA, 22 nt, Artificial Sequence
  Description of Artificial Sequence: Synthetic antagomir to hsa-miR-200a-3p sequence
acaucguuaccagacaguguua

SEQ ID NO: 12 - RNA, 22 nt, Artificial Sequence
  Description of Artificial Sequence: Synthetic antagomir to hsa-miR-200b-3p sequence
ucaucauuaccaggcaguauua

SEQ ID NO: 13 - RNA, 20 nt, Artificial Sequence
  Description of Artificial Sequence: Synthetic antagomir to hsa-miR-200c-3p sequence
uccaucacccggcaguauua

SEQ ID NO: 14 - RNA, 19 nt, Artificial Sequence
  Description of Artificial Sequence: Synthetic antagomir to hsa-miR-429-3p sequence
acgguuccagacaguauua

SEQ ID NO: 15 - DNA, 20 bp, Artificial Sequence
  Description of Artificial Sequence: Synthetic CRISPR Cas 9 guide RNA G1 to human miR-141 sequence
ggccggccgacagagaacta

SEQ ID NO: 16 - DNA, 20 bp, Artificial Sequence
  Description of Artificial Sequence: Synthetic CRISPR Cas 9 guide RNA G2 to human miR-141 sequence
ctgtactggaagatggaccc

SEQ ID NO: 17 - DNA, 20 bp, Artificial Sequence
  Description of Artificial Sequence: Synthetic CRISPR Cas 9 guide RNA G3 to human miR-141 sequence
tgtactggaagatggaccca

SEQ ID NO: 18 - RNA, 22 nt, Mus musculus
  mmu-miR-141-3p mature micro-RNA
uaacacugucugguaaagaugg

SEQ ID NO: 19 - RNA, 22 nt, Mus musculus
  mmu-miR-200a-3p mature micro-RNA
uaacacugucugguaacgaugu

SEQ ID NO: 20 - RNA, 23 nt, Mus musculus
  mmu-miR-200b-3p mature micro-RNA
uaauacugccggguaaugaugga

SEQ ID NO: 21 - RNA, 22 nt, Mus musculus
  mmu-miR-200c-3p mature micro-RNA
uaauacugccggguaaugauga

SEQ ID NO: 22 - RNA, 22 nt, Mus musculus
  mmu-miR-429 mature micro-RNA
uaauacugucugguaaugccgu

SEQ ID NO: 23 - DNA, 118 bp, Homo sapiens
  MIR141 subsequence including miR-141 stem loop
gcaccgtagttctctgtcggccggccctgggtccatcttccagtacagtgttggatggtctaattgtgaagctcctaacactgtctggtaaagatggctcccgggtgggttctctcgg